(19) United States
(12) Patent Application Publication
(10) Pub. No.: US 2026/0023002 A1
(43) Pub. Date: Jan. 22, 2026

(54) ANALYSIS METHOD, NON-TRANSITORY COMPUTER-READABLE STORAGE MEDIUM, AND ANALYSIS DEVICE

(71) Applicants: NHK SPRING CO., LTD., Yokohama-shi (JP); NATIONAL UNIVERSITY CORPORATION TOKAI NATIONAL HIGHER EDUCATION AND RESEARCH SYSTEM, Nagoya-shi (JP)

(72) Inventors: Shintaro KUMAI, Yokohama-shi (JP); Keita TAKAHASHI, Yokohama-shi (JP); Kosuke ONUMA, Yokohama-shi (JP); yuichiro YAMAUCHI, Yokohama-shi (JP); Yoshitaka ADACHI, Nagoya-shi (JP)

(21) Appl. No.: 19/341,609

(22) Filed: Sep. 26, 2025

Related U.S. Application Data
(63) Continuation of application No. PCT/JP2024/012600, filed on Mar. 28, 2024.

(30) Foreign Application Priority Data
Mar. 31, 2023 (JP) 2023-058185

(51) Int. Cl. G01N 3/40; G01N 33/204

(52) U.S. Cl. CPC G01N 3/40; G01N 33/204

(57) ABSTRACT

An analysis method includes providing first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and obtaining first property information on the first spring steel from the learning model.

### CROSS REFERENCE TO RELATED APPLICATIONS

This application is a Continuation of International Patent Application No. PCT/JP2024/012600, filed on Mar. 28, 2024 which claims the benefit of priority to Japanese Patent Application No. 2023-58185, filed on Mar. 31, 2023 the entire contents of which are incorporated herein by reference.

### FIELD

An embodiment of the present invention is an analysis method, a program, and an analysis device for predicting the properties of steel.

### BACKGROUND

In the production and development of steel, in order to easily and quickly evaluate the performance of the materials, it is required to accurately predict the properties from the metal microstructure of a manufactured steel material. Japanese laid-open patent publication No. 2022-136745 discloses a program for predicting the properties of steel by performing machine learning using map data in which values are mapped to elements having location information such as an optical microscope image and a scanning electron microscope image.

### SUMMARY

An analysis method according to an embodiment of the present invention includes providing first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and obtaining first property information on the first spring steel from the learning model.

A non-transitory computer-readable storage medium having a program stored thereon according to an embodiment of the present invention, the program causing a computer to provide first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and obtain first property information on the first spring steel from the learning model.

An analysis device according to an embodiment of the present invention includes a providing unit providing first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and an acquisition unit obtaining first property information on the first spring steel from the learning model.

### BRIEF DESCRIPTION OF DRAWINGS

FIG. 1 is a block diagram showing an analysis device according to an embodiment of the present invention.

FIG. 2 is a block diagram showing various types of information stored in a storage unit.

FIG. 3 is a block diagram illustrating a learning model generation function according to an embodiment of the present invention.

FIG. 4 is a flowchart illustrating a learning model generation method according to an embodiment of the present invention.

FIG. 5 is a block diagram illustrating an analysis function according to an embodiment of the present invention.

FIG. 6 is a flowchart illustrating an analysis method according to an embodiment of the present invention.

### DESCRIPTION OF EMBODIMENTS

In the case of Patent Literature 1, since each map data of the steel material is labeled and machine learning is performed, it is not possible to predict the properties of the new material. Further, it is difficult to accurately quantify dislocation density and solid-solution carbon content, which affect the material properties of steel, from the map data. In particular, it is difficult to predict the time-dependent material properties, such as the strain and fatigue of steel, from the map data.

In view of the above problems, an object of an embodiment of the present invention is to accurately predict the properties of steel.

Hereinafter, embodiments of the present invention disclosed in the present application will be described with reference to the drawings. However, the present invention can be implemented in various forms without departing from the gist thereof, and is not to be construed as being limited to the description of the embodiments exemplified below.

Further, in the present specification and the drawings, elements having functions similar to those described with respect to the above-described drawings are denoted by the same reference signs, and redundant descriptions thereof may be omitted.

In the present specification and claims, each term is defined as follows.

Spring steel generally refers to carbon-based, silicon-manganese-based, manganese-chromium-based, chromium-vanadium-based steels, and the like. In addition, the spring steel is not limited to the steels described above, and may include steel applicable to a spring.

Material information includes information obtained from the surface of steel and information obtained from the interior of spring steel. The material information includes information obtained from the surface of spring steel (hereinafter, referred to as surface information) and information obtained from the interior of spring steel (hereinafter, also referred to as internal information). In this case, the information obtained from the surface of spring steel includes respective parameters of crystal grain size, carbide morphology, retained γ, and surface roughness. In addition, the parameters obtained from the interior of spring steel include carbide morphology, dislocation density, solid-solution carbon content, retained γ, dislocation character, residual stress, and inclusion distribution.

The material properties are values representing the mechanical properties and time-dependent properties of spring steel. The mechanical properties include hardness, tensile properties, and toughness, and the time-dependent properties include strain and fatigue. The strain and fatigue may be actual measurements or may be qualitative evaluations.

### Configuration of Analysis Unit

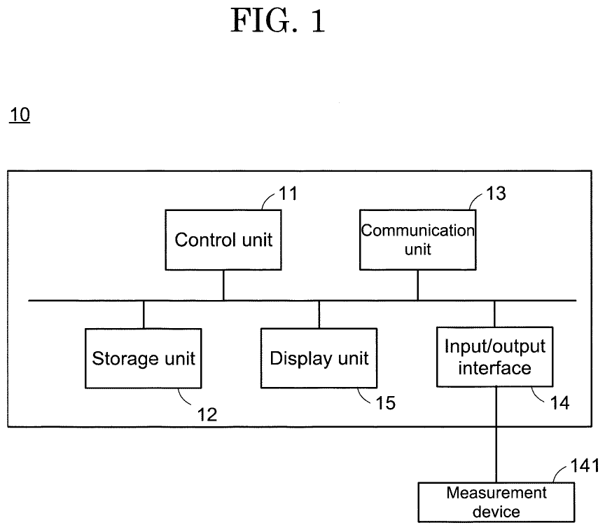

In the present embodiment, an analysis device, an analysis method, and a program according to an embodiment will be described with reference to FIG. 1 to FIG. 6.

### 1. Overview of Analysis Unit

In the analysis unit according to an embodiment of the present invention, property information on spring steel is predicted from the material information on spring steel by using a learning model that has learned a relationship between the material information on spring steel and the property information on spring steel in advance. Not only the parameters obtained from map data, such as SEM observations of the surface of spring steel, but also parameters obtained from information related to a periodic structure inside spring steel obtained from XRD, which quantifies finer structures, are used as the material information. This makes it possible to accurately predict material properties such as hardness and tensile properties of spring steel. Further, it is possible to accurately predict the material properties of spring steel, which has been difficult to predict in the past. In addition, by predicting the material properties of newly manufactured spring steel, it is possible to shorten the development period and reduce the cost.

### 2. Configuration of Analysis Unit

FIG. 1 is a block diagram showing a configuration of an analysis device 10 according to the present embodiment. The analysis device includes a control unit 11, a storage unit 12, a communication unit 13, an input/output interface 14, and a display unit 15. The analysis device 10 is not limited to the inclusion of all of these configurations.

The control unit 11 is an example of a computer including a processor (calculation processing circuit) such as a CPU (Central Processing Unit) and a storage device such as a RAM. The control unit 11 executes various programs stored in the storage unit 12 by a processor to implement various functions in the analysis device 10. For example, the control unit 11 implements an analysis function for predicting the property information on spring steel by executing an analysis program. In addition, the control unit 11 executes a learning model generation program to implement generation of a learning model for learning a relationship between the material information and the material properties of spring steel.

The storage unit 12 is a storage device such as a non-volatile memory or a hard disk drive. The storage unit 12 stores a material database related to the spring steel. The storage unit 12 stores the learning model that has learned the relationship between the material information on spring steel and the property information on spring steel. The storage unit 12 is a non-transitory computer-readable storage medium, and a program may be recorded in the storage medium. The program is the learning model generation program and the analysis program. The storage unit 12 stores, in the control unit 11, information such as a program and parameters required to execute the program. The storage unit 12 will be described in detail later. Further, the storage unit 12 is illustrated as a single storage device in FIG. 1, but may be composed of a plurality of storage devices.

The communication unit 13 is connected to a network NW under the control of the control unit 11 to transmit and receive data to and from other computers, servers, or other devices connected to the network NW.

For example, the input/output interface 14 may be connected to an input means such as a keyboard and a mouse. In addition, various measurement devices 141 for obtaining the material information and property information on spring steel may be connected to the input/output interface 14. The measurement device 141 includes an optical microscope, a Scanning Electron Microscope (SEM), an Electron Backscatter Diffraction (EBSD), a Transmission Electron Microscope (TEM), an X-ray Diffraction (XRD), a surface roughness meter (contact or non-contact), an Atomic Force Microscope (AFM), a Small Angle X-ray Scattering, an Atomic Probe Tomography (APT), and the like. The analysis device 10 may be connected to the measurement device 141 via the communication unit 13. Alternatively, the analysis device 10 may be connected to the measurement device 141 and a server connected to the measurement device 141 via the communication unit 13.

For example, the display unit 15 is a display device such as a liquid crystal display panel or an EL display panel. The display unit 15 may be a display device integrally connected to the inside of the housing forming the analysis device 10. Alternatively, the display unit 15 may be an external display device connected to the input/output interface 14. The display unit 15 displays a display screen related to an acquisition unit, which is a functional block of the analysis function described later.

### 3. Configuration of Storage Unit

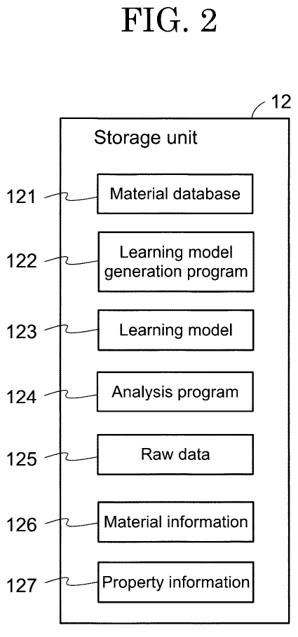

FIG. 2 is a block diagram showing various types of information stored in the storage unit 12. The storage unit 12 includes a material database 121, a learning model generation program 122, a learning model 123, an analysis program 124, raw data 125, material information 126, and property information 127. The storage unit 12 is not limited to storing all of these configurations, and may store a part of these configurations. The various types of information in FIG. 2 are not limited to being stored in one storage device, and may be stored in a plurality of storage devices in a distributed manner.

The material database 121, the learning model generation program 122, the learning model 123, the analysis program 124, the raw data 125, the material information 126, and the property information 127 may be provided in a state of being recorded in a computer-readable recording medium such as a magnetic recording medium, an optical recording medium, a magneto-optical recording medium, or a semiconductor memory. In this case, the analysis device 10 only needs to be equipped with a device for reading the recording medium.

### 3-1. Raw Data

The raw data 125 obtained from various measurement devices 141 is stored in the storage unit 12. Image data obtained from an optical microscope, SEM, SEM/EBSD, TEM, or the like may be stored in the storage unit 12 as the raw data 125. In addition, an XRD spectrum obtained from XRD may be stored in the storage unit 12 as the raw data 125. Further, surface roughness data obtained from a surface roughness meter and AFM may be stored in the storage unit 12 as the raw data 125. The data stored as the raw data 125 is not limited to the above-described data, and may be data obtained from various measurement devices 141.

### 3-2. Material Information

The material information 126 stores the surface information and the internal information on spring steel. The surface information and the internal information may be converted into various parameters by analyzing the raw data 125 stored in the storage unit 12. The surface information and the internal information may be obtained from the measurement device 141. The surface information and the internal information may be the surface information and the internal information on the analyzed spring steel, or may be the surface information and the internal information on spring steel of an analysis target. Alternatively, the surface information and the internal information may be stored in the material database 121 at a later time in order to be used as training data of the learning model.

The surface information refers to observation information on the surface of spring steel. The observation information on the surface of spring steel is obtained from image data or shape data such as SEM, SEM/EBSD, surface roughness meter, and AFM. In addition, the data obtained from the interior of spring steel can be obtained from XRD, X-ray small angle scattering, atomic probe tomography, optical microscope, SEM/EBSD, and the like. The information obtained from the interior of spring steel may be image data obtained by observing the inside of spring steel by SEM.

The parameter of the crystal grain size can be obtained as the surface information by analyzing the image data obtained by observing the surface of spring steel using the optical microscope or SEM/EBSD. Examples of the parameter representing the crystal grain size include circle equivalent diameter (um), circularity, and aspect ratio. The circle equivalent diameter (um) is expressed using an arithmetic mean, weighted mean, maximum value, minimum value, median value, and standard deviation. In addition, the circularity is a value obtained by dividing the circle equivalent circumference by the actual circumference. The aspect ratio is the ratio of the crystal grain size in the longitudinal direction (rolling direction) of steel to the crystal grain size in the width direction (direction perpendicular to the longitudinal direction) of steel.

The carbide morphology can be obtained as the surface information by analyzing the image data obtained by observing the surface of spring steel using SEM. In addition, the carbide morphology can be obtained as the internal information by analyzing the profile of the scattering angle and the scattering intensity of the X-ray obtained by using X-ray small angle scattering. Examples of the parameter representing the carbide morphology include a circle equivalent diameter (μm), a number density, an aspect ratio, an area ratio, and a volume ratio. The circle equivalent diameter (μm) is expressed using an arithmetic mean, weighted mean, maximum value, minimum value, median value, and standard deviation. The number density (number/μm²) refers to the number of grains of carbides in a unit area. In addition, the number density (number/μm³) refers to the number of grains of carbides in a unit volume. The aspect ratio refers to the ratio (ellipticity) between the major axis and the minor axis in the size of the carbide. The area ratio refers to the area of carbides in an evaluation range. The volume ratio refers to the volume of carbides in the evaluation range. In this case, parameters of the circle equivalent diameter, the number density (the number of grains of carbides in a unit area), the aspect ratio, and the area ratio are used as the surface information. The parameters of the number density (the number of grains of carbides in the unit volume) and the volume ratio are used as the internal information.

The surface roughness can be obtained by analyzing the image data obtained by measuring the surface of spring steel using AFM or the shape data and the image data obtained by measuring the surface of spring steel using the surface roughness meter (contact or non-contact). Examples of the parameter representing the surface roughness include an arithmetic mean roughness Ra, maximum height roughness Rz (axial, circumference, 45° direction), and the like. In this case, a direction perpendicular to the axis or forming 45° with respect to the line longitudinal direction (or rolling direction) as an axis is referred to as a circumferential direction or a 45° direction, respectively.

The dislocation density can be obtained as the internal information by analyzing the XRD spectrum inside the spring steel using XRD. The dislocation density (m⁻²) is used as a parameter representing the dislocation density. In addition, image data obtained by observing the cross section of spring steel may be analyzed as the internal information using TEM. For example, the dislocation density can be quantified by measuring dislocation lines in the image observed using TEM and dividing by an observation range (field size×sample thickness).

The solid-solution carbon content can be obtained as the internal information by analyzing the XRD spectrum of spring steel using XRD. The relationship between the lattice constant of Fe in steel and the solid-solution carbon content is generally known. In the case of using XRD, the solid-solution carbon content can be obtained by calculating the lattice constant by XRD and then converting the calculated amount into the solid-solution carbon content. Further, in the case of using the electrical resistance measurement method, the solid-solution carbon content can be indirectly obtained by preparing a known comparative material by using the correlation between the electrical resistance and the solid-solution carbon content. In addition, APT is used to analyze atomic-level 3D mappings to obtain the solid-solution carbon content within the evaluation range. The solid-solution carbon content (wt %) is used as a parameter representing the solid-solution carbon content.

The retained γ can be obtained as the internal information by analyzing the XRD spectrum of spring steel using XRD. In addition, the retained γ can be obtained as the surface information by analyzing the image data obtained by observing the surface of spring steel using SEM/EBSD. The retained γ amount (%) is used as a parameter representing the retained γ.

The dislocation character can be obtained as the internal information by analyzing the XRD spectrum of spring steel using XRD. The ratio (%) of edge dislocations and helical dislocations is used as a parameter representing the dislocation character.

The residual stress can be obtained as the internal information by analyzing the XRD spectrum of spring steel using XRD. Surface residual stress (MPa), Crossing point depth (mm), Maximum residual stress (MPa), and Maximum residual stress depth (mm) are used as the parameter representing residual stress.

The inclusion distribution can be quantified by observing the fracture surface after fatigue testing or hydrogen-charged tensile testing. For example, the inclusion distribution can be obtained as the internal information by observing the cross section by SEM, thereby obtaining the image data. Depth (mm) and size (mm) are used as a parameter representing the inclusion distribution. The depth (mm) and size (mm) can be obtained by statistically processing the mean, variance, maximum, and minimum.

Dislocation density, solid-solution carbon content, retained γ, dislocation character, and residual stress are difficult to quantify from the images observed using SEM. In the present embodiment, dislocation density, solid-solution carbon content, retained γ, dislocation character, and residual stress can be quantified by using XRD or the like capable of obtaining information related to the periodic structure of the inside of spring steel. As a result, it is possible to accurately predict values such as strain and fatigue, which represent the time-dependent properties of spring steel, which has been difficult to predict in the past.

### 3-3. Property Information

The property information 127 representing the material properties of spring steel includes at least one of the mechanical properties and the time-dependent properties. The mechanical properties include hardness, tensile properties, and toughness, and the time-dependent properties include strain and fatigue. Parameters obtained by measuring the hardness, tensile properties, toughness, strain, and fatigue of spring steel may be stored as the property information 127, or parameters predicted by the learning model may be stored as the property information 127. The respective parameters of the hardness, tensile properties, toughness, strain, and fatigue of spring steel are obtained by the method described below.

For example, the hardness of spring steel can be obtained by the Vickers hardness test (JIS Z2244) as Vickers Hardness (HV). The Rockwell Hardness Test (JIS Z2245), the Brinell Hardness Test (JIS 2243), and the Shore Hardness Test (JIS Z2246) may be used as the method for evaluating hardness. For example, the tensile properties are obtained by the tensile testing (JIS Z2241) as tensile properties (MPa), 0.2% yield strength (MPa), elongation (%), and contraction (%). In addition, tensile properties and yield strength are calculated from SS diagrams obtained in the test, and elongation and contraction are calculated from the test piece dimensions before and after the test. For example, the toughness can be obtained by the Charpy Impact Test (JIS Z2242) as a Charpy Impact Value (J/cm²). JIS stands for Japanese Industrial Standards.

For example, fatigue can be obtained by measuring the fatigue limit (MPa) using the 14S-N test method (e.g., as described in JSME S 002). For example, creep strain (%) can be obtained as strain by performing a creep test at a predetermined temperature, stress, and time. In addition, the method for measuring hardness, tensile properties, toughness, fatigue, and strain is not limited to the method described above, and known methods may be used.

### 3-4. Material Database

The material database 121 stores the material information and property information on spring steel measured in advance. A unique identifier is assigned to each spring steel, and the identifier is associated with the material information 126 and the property information 127 and stored. The material information 126 and the property information 127 stored in the material database 121 are used as the training data of the learning model. All or part of the material information 126, the parameters representing crystal grain size, carbide morphology, surface roughness, dislocation density, solid-solution carbon content, retained γ, dislocation character, residual stress, and inclusion distribution corresponding to spring steel are stored. In addition, all or part of the above-described parameters representing hardness, tensile properties, toughness, strain, and fatigue are stored as the property information 127.

A part of the material information 126 and a part of the property information 127 of spring steel stored in the material database 121 may also be used to generate the learning model 123. However, the larger the number of parameters representing the material information associated with one spring steel, or the larger the number of parameters representing the property information, the higher the prediction accuracy of the property information on spring steel, which is preferable.

The learning model generation program 122 is a program for generating the learning model 123 used in the analysis program 124. The learning model 123 learns the relationship between the material information on spring steel and the property information on spring steel in advance. The analysis program 124 is a program for obtaining the property information on spring steel by providing the obtained material information on spring steel to the learning model 123. The learning model generation program 122, the learning model 123, and the analysis program 124 will be described in detail later.

### 4. Learning Model Generation Function

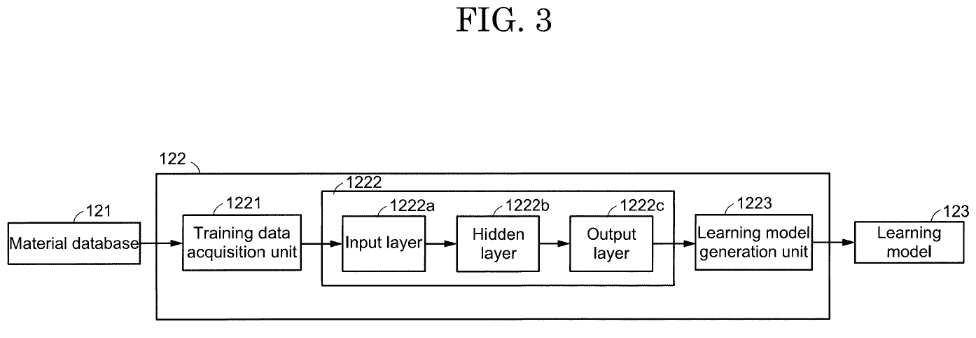

FIG. 3 is a block diagram illustrating a learning model generation function according to an embodiment of the present invention. The learning model generation function includes a training data acquisition unit 1221, a neural network 1222, and a learning model generation unit 1223. In the generation stage of the learning model, the neural network 1222 that inputs the material information on spring steel and outputs the property information on spring steel is generated. That is, by generating the neural network 1222 in advance, it is possible to predict the property information on spring steel simply by obtaining the parameters of the material information on spring steel in advance.

The training data acquisition unit 1221 obtains a relationship between the material information 126 and the property information 127 of spring steel stored in the material database 121 as training data.

The learning model generation unit 1223 uses the training data obtained by the training data acquisition unit 1221 to generate the neural network 1222 that inputs the material information on spring steel and outputs the property information on spring steel. Regarding the material information and the property information used for the training data, respective parameters of crystal grain size, carbide morphology, dislocation density, solid-solution carbon content, retained γ, dislocation character, residual stress, surface roughness, and inclusion distribution are included as the material information, and respective parameters of hardness, tensile properties, toughness, strain, and fatigue are included as the property information. All or a part of these parameters may be used as the training data as the parameters representing the material information and the property information.

The neural network 1222 includes an input layer 1222a, a hidden layer 1222b, and an output layer 1222c. The material information on spring steel is input to the input layer 1222a. The material information on spring steel is output to the output layer 1222c. Parameters (coupling factor between nodes) are learned in the hidden layer 1222b by using the training data including the material information on spring steel and the property information on spring steel. As a result, the learning model 123 can be generated.

The learning model 123 generated by the learning model generation unit 1223 is stored in the storage unit 12.

### 5. Learning Model Generation Method

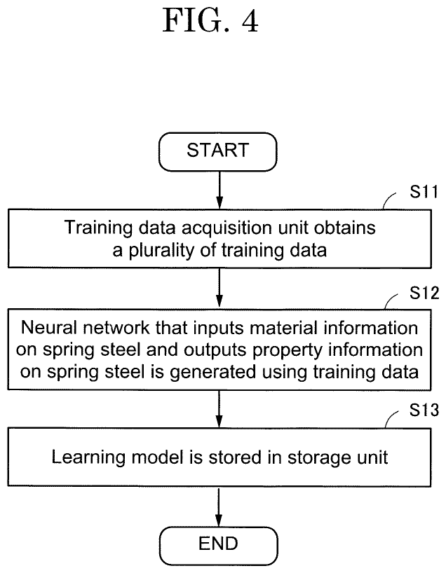

FIG. 4 is a flowchart illustrating a learning model generation method according to an embodiment of the present invention. The learning model generation function is implemented by the control unit 11 executing the learning model generation program 122. One or all of the configurations for realizing the learning model generation function described below may be implemented by hardware.

When the learning model generation program 122 is started by the control unit 11 in step S11, the training data acquisition unit 1221 obtains the relationship between the material information and the property information on spring steel stored in the material database 121 as the training data.

In step S12, the neural network that inputs the material information on spring steel and outputs the property information on spring steel is generated using the training data.

In step S13, the generated learning model 123 is stored in the storage unit 12.

As a result, the operation of the learning model 123 generation program is completed. The generated learning model 123 may be implemented in a general-purpose computer or communication terminal, or may be downloaded as software or an application, or may be distributed while being stored in a storage medium.

The more parameters representing the material information associated with spring steel and the more parameters representing the property information, the higher the accuracy of the property information on spring steel obtained from the learning model 123.

In addition, the learning model 123 performs relearning for a predetermined period, and updates the learning model 123. The learning model 123 may relearn the material information and the property information on spring steel as the training data based on past achievements. That is, the learning model 123 can further improve the prediction accuracy of the property information on spring steel by the previous learning model 123 by increasing the data in which the material information and the property information for each spring steel are associated, which are accumulated in the material database. In addition, the update timing of the learning model 123 is not limited to a predetermined period, and may be executed at a timing at which a predetermined number of pieces of training data are accumulated.

### 6. Analysis Function

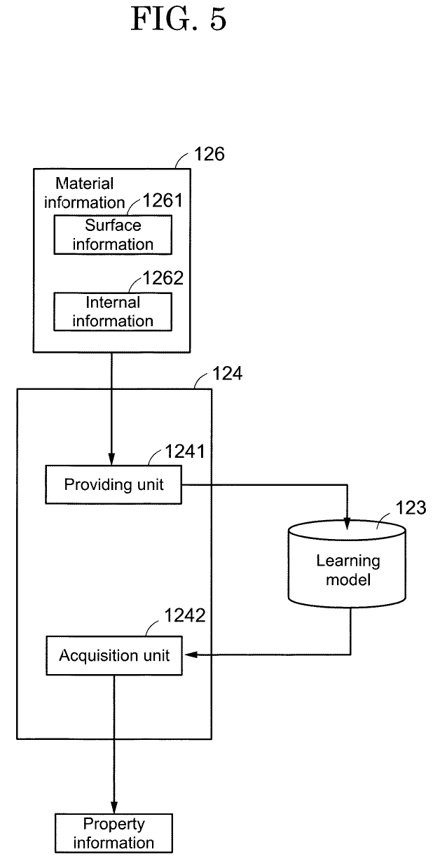

FIG. 5 is a block diagram showing an analysis function according to an embodiment of the present invention. The analysis function includes a providing unit 1241 and an acquisition unit 1242.

The providing unit 1241 obtains material information including at least one of the surface information and the internal information on spring steel. The providing unit 1241 may obtain the material information 126 (surface information 1261 and internal information 1262) of an analysis target from the material information 126 stored in the storage unit 12, or may obtain the material information (the surface information 1261 and the internal information 1262) of an analysis target from the material database 121. The providing unit 1241 provides the material information 126 of an analysis target to the learning model 123.

In the learning model 123, when the material information 126 of an analysis target is obtained, the material information 126 of an analysis target is input to the input layer 1222a of the neural network 1222. Then, in the hidden layer 1222b, a calculation is performed based on a parameter (the coupling factor between the nodes), and the property information is output from the output layer 1222c.

The acquisition unit 1242 obtains the predicted property information from the learning model 123, and outputs the property information.

In addition, a previous processing unit may be provided for obtaining image data or measurement data from the measurement device 141 and generating surface information or internal information from the image data or measurement data without storing it in the storage unit 12.

### 7. Analysis Method

The function of analyzing the material properties of spring steel is implemented by the control unit 11 executing the analysis program 124.

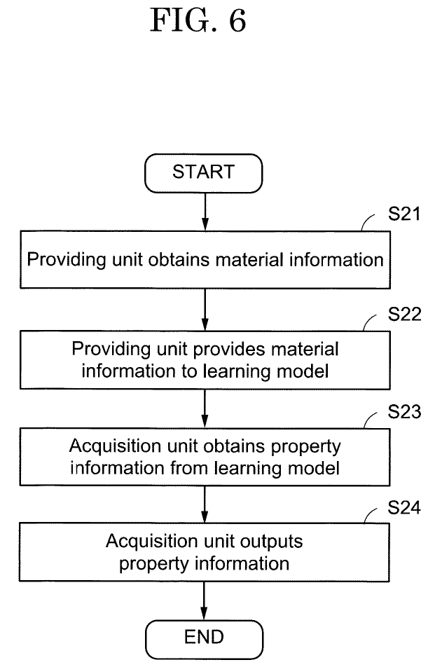

FIG. 6 is a flowchart showing an analysis method according to an embodiment of the present invention.

When the analysis program 124 is started by the control unit 11 in step S21, the providing unit 1241 obtains the material information including the surface information and the internal information.

In step S22, the providing unit 1241 provides the information on spring steel to the learning model 123.

In step S23, the acquisition unit 1242 obtains the property information from the learning model 123.

In step S24, the acquisition unit 1242 outputs the property information obtained from the learning model 123.

As described above, the operation of the analysis program is completed.

Next, the material information affecting various material properties is shown in Table 1. Items marked with “0” in Table 1 are items having a large degree of contribution in characteristic prediction.

TABLE 1

| | Material properties | | | | |
|---|---|---|---|---|---|
| Material | | Tensile | Tough- | | |
| information | Hardness | properties | ness | Strain | Fatigue |
| Crystal grain size | ◯ | ◯ | ◯ | ◯ | ◯ |
| Carbide morphology | ◯ | ◯ | ◯ | ◯ | ◯ |
| Dislocation density | ◯ | ◯ | ◯ | ◯ | ◯ |
| Solid-solution | ◯ | ◯ | ◯ | ◯ | ◯ |
| carbon content | | | | | |
| Retained γ | | | ◯ | ◯ | ◯ |
| Dislocation character | | | | ◯ | ◯ |
| Residual stress | | | | ◯ | ◯ |
| Surface roughness | | | | | ◯ |
| Inclusion distribution | | | | | ◯ |

As shown in Table 1, in order to obtain the property information including hardness, tensile properties, toughness, strain, and fatigue of spring steel, parameters representing crystal grain size, carbide morphology, dislocation density, and solid-solution carbon content may be provided to the learning model 123.

In order to improve the accuracy of the property information on toughness, a parameter representing retained γ is provided to the learning model 123 in addition to the parameters representing crystal grain size, carbide morphology, dislocation density, and solid-solution carbon content, thereby improving the accuracy of predicting toughness.

Further, in order to improve the accuracy of the property information on dislocation, at least one of the parameters representing retained γ, dislocation character, and residual stress is supplied to the learning model 123, in addition to the parameters representing crystal grain size, carbide morphology, dislocation density, and solid-solution carbon content, thereby improving the accuracy of predicting strain.

Further, in order to improve the accuracy of the property information on fatigue, at least one of the parameters representing retained γ, dislocation character, residual stress, surface roughness, and inclusion distribution is supplied to the learning model 123, in addition to the parameters representing crystal grain size, carbide morphology, dislocation density, and solid-solution carbon content, thereby improving the accuracy of predicting fatigue.

According to an embodiment of the present disclosure, microstructural information on spring steel, such as dislocation density and solid-solution carbon content, which are not captured by map data, such as a SEM observation of the surface of spring steel, is quantified using XRD and the like. As a result, it is possible to predict not only hardness and tensile properties but also time-dependent properties such as toughness, strain, and fatigue as the material properties.

In the present embodiment, parameters obtained by quantifying dislocation density, solid-solution carbon content, retained γ, and residual stress from the XRD spectrum, which are difficult to obtain from the image data obtained from the SEM observation of the surface of spring steel, are used as inputs to the learning model 123. As a result, it is possible to predict time-dependent property information, such as strain and fatigue, which is conventionally difficult to predict. As a result, it is possible to improve the accuracy of predicting the property information on the developed spring steel.

### 8. Modification

The present invention is not limited to the above-described embodiments, and includes various other modifications. For example, the above-described embodiments have been described in detail for the purpose of illustrating the present invention in an easy-to-understand manner, and are not necessarily limited to those having all the described configurations. Other configurations may be added, deleted, or substituted for some of the configurations of the embodiments and some of the modifications described below. Hereinafter, a modification will be described.

(1) In the present embodiment, an example has been described in which the learning model 123 is generated by using, as the material information, parameters representing crystal grain size, carbide morphology, surface roughness, dislocation density, solid-solution carbon content, retained γ, dislocation character, residual stress, and inclusion distribution, and using, as the property information, parameters representing hardness, tensile properties, strain, and fatigue, but an embodiment of the present invention is not limited to this. The learning model 123 may be generated for each material property that needs to be predicted. In this case, the material information input to the learning model 123 may be selected based on Table 1.

For example, in order to predict the hardness or tensile properties of spring steel as the material properties, parameters representing crystal grain size, carbide morphology, dislocation density, and solid-solution carbon content are used as inputs to the learning model 123. That is, the learning model 123 for predicting hardness or tensile properties may be generated by inputting parameters representing crystal grain size, carbide morphology, dislocation density, and solid-solution carbon content.

Further, in order to predict the toughness of spring steel as the material properties, parameters representing crystal grain size, carbide morphology, dislocation density, solid solution carbon content, and retained γ are used as inputs to the learning model 123. In other words, the learning model 123 for predicting toughness may be generated by inputting parameters representing crystal grain size, carbide morphology, dislocation density, solid-solution carbon content, and retained γ.

Further, in order to predict the strain of spring steel as the material properties, parameters representing crystal grain size, carbide morphology, dislocation density, solid-solution carbon content, retained γ, dislocation character, and residual stress are used as inputs to the learning model 123. In other words, the learning model 123 for predicting strain may be generated by inputting parameters representing crystal grain size, carbide morphology, dislocation density, solid-solution carbon content, retained γ, dislocation character, and residual stress.

Further, in order to predict the fatigue of spring steel, parameters representing crystal grain size, carbide morphology, dislocation density, solid-solution carbon content, retained γ, dislocation character, residual stress, surface roughness, and inclusion distribution are used as inputs to the learning model 123. In other words, the learning model 123 for predicting fatigue may be generated by inputting parameters representing crystal grain size, carbide morphology, dislocation density, solid-solution carbon content, retained γ, dislocation character, residual stress, surface roughness, and inclusion distribution.

(2) In the present embodiment, the parameters representing crystal grain size, carbide morphology, surface roughness, dislocation density, solid-solution carbon content, retained γ, dislocation character, residual stress, and inclusion distribution are used as the material information. However, an embodiment of the present invention is not limited to this. The above-described material information may further include information on the mechanical properties among the condition information at the time of manufacturing spring steel, the element information forming spring steel, and the property information.

A heat treatment method of spring steel is exemplified as the condition information at the time of manufacturing spring steel. The conditions for quenching and tempering can be used as input parameters for the main heat treatment method of spring steel. For example, in the case of quenching spring steel, temperature, time, heating means (furnace heating, induction heating, or current heating, etc.) can be used as a parameter, and in the case of tempering spring steel, temperature, time, heating means (furnace heating, induction heating, or current heating, etc.) can be used as parameters. In addition, conditions for annealing or normalization may be used as input parameters for the heat treatment method. The temperature, time, and heating means may also be used as parameters in the case of using the conditions for annealing or normalization as the input parameters. That is, for the condition information at the time of manufacturing spring steel, at least one of quenching, tempering, annealing, and normalization may be selected as the heat treatment method, and at least one of time, temperature, and heat treatment may be selected among the heat treatment methods.

Typical examples of the elements forming spring steel include Ni, Mn, C, Si, Cu, Cr, Ti, V, Mo, and B. In addition, for example, the parameter of the element forming spring steel may be the content (wt %) of elements in spring steel or a ratio. Further, the analytical method for the elements forming spring steel includes a spark optical emission spectroscopy (OES; Optical Emission Spectrometer). Alternatively, the parameter of the element forming spring steel may be a value described in a mill sheet which is a document that certifies the quality of the steel (steel inspection certificate). Alternatively, information in a database such as Total Materia (Total Materia AG) may be referred to.

At least one of the hardness, tensile properties, and toughness of spring steel of the property information may be included as information on the mechanical properties. The material information may include the hardness among the property information as information on the mechanical properties. The hardness, tensile properties, and toughness of spring steel relate to the mechanical properties, but may be used as parameters to characterize spring steel because testing can be performed in a short time compared with strain and fatigue. The description of the property information may be referred to with regards to the hardness, tensile properties, and toughness of spring steel. In addition, the hardness of spring steel may be used as information obtained from the surface of spring steel.

In the case of predicting the hardness or tensile properties of spring steel as the material properties, at least one of the parameters representing the composition of spring steel and the condition at the time of manufacturing spring steel may be used as inputs to the learning model 123, in addition to the parameters representing crystal grain size, carbide morphology, dislocation density, and solid-solution carbon content.

Further, in the case of predicting the toughness of spring steel as the material properties, at least one of the parameters representing the hardness of spring steel, the composition of spring steel, and the condition at the time of manufacturing spring steel may be used as the inputs to the learning model 123, in addition to the parameters representing crystal grain size, carbide morphology, dislocation density, solid-solution carbon content, and retained γ.

Further, in the case of predicting the strain of spring steel as the material properties, at least one of the parameters representing the hardness of spring steel, the composition of spring steel, and the condition at the time of manufacturing spring steel may be used as the inputs to the learning model 123, in addition to the parameters representing crystal grain size, carbide morphology, dislocation density, solid-solution carbon content, retained γ, dislocation character, and residual stress.

Further, in the case of predicting the fatigue of spring steel as the material properties, at least one of the parameters representing the hardness of spring steel, the composition of spring steel, and the condition at the time of manufacturing spring steel may be used as the inputs to the learning model 123, in addition to the parameters representing crystal grain size, carbide morphology, dislocation density, solid-solution carbon content, retained γ, dislocation character, residual stress, surface roughness, and inclusion distribution.

That is, the learning model 123 includes learning a relationship between the material information including information obtained from the surface of spring steel, information obtained from the interior of spring steel, and the condition information at the time of manufacturing spring steel, and the property information. In addition, the information obtained from the interior of spring steel may include the composition information on spring steel. The learning model 123 with high prediction accuracy can be generated by selecting and learning the above-described parameters with respect to a characteristic to be predicted.

The learning model 123 includes learning a relationship between the material information, including information obtained from the surface of spring steel, information obtained from the interior of spring steel, and the mechanical properties of spring steel, and the property information, including time-dependent properties of spring steel. The learning model 123 with high prediction accuracy can be generated by selecting and learning the above-described parameters with respect to a characteristic to be predicted.

Each of the embodiments described above as an embodiment of the present disclosure can be appropriately combined and implemented as long as no contradiction is caused. Further, the addition, deletion, or design change of components as appropriate by those skilled in the art based on each embodiment are also included in the scope of the present disclosure as long as they are provided with the gist of the present disclosure.

Further, it is understood that, even if the effect is different from those provided by each of the above-described embodiments, the effect obvious from the description in the specification or easily predicted by persons ordinarily skilled in the art is apparently derived from the present disclosure.

What is claimed is:

1. An analysis method comprising:
providing first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and
obtaining first property information on the first spring steel from the learning model.

2. The analysis method according to claim 1, wherein
the second information obtained from the interior of the spring steel includes third information related to a periodic structure of the interior of the spring steel.

3. The analysis method according to claim 2, wherein
the third information related to a periodic structure is obtained from an XRD spectrum.

4. The analysis method according to claim 1, wherein
the second information obtained from the interior of the spring steel is at least one of dislocation density, solid-solution carbon content, retained γ, dislocation character, residual stress, and inclusion distribution.

5. The analysis method according to claim 1, wherein
the first information obtained from the surface of the spring steel includes observation information on the surface of the spring steel.

6. The analysis method according to claim 1, wherein
the first information obtained from the surface of the spring steel is at least one of crystal grain size, carbide morphology, and surface roughness.

7. The analysis method according to claim 1, wherein
the material properties of the spring steel are at least one of mechanical properties and time-dependent properties.

8. The analysis method according to claim 7, wherein
the mechanical properties include hardness, tensile properties, and toughness.

9. The analysis method according to claim 7, wherein
the time-dependent properties include strain and fatigue.

10. The analysis method according to claim 1, wherein
the learning model learns a relationship between the material information, the material information including the first information obtained from the surface of the spring steel, the second information obtained from the interior of the spring steel, condition information at the time of manufacturing the spring steel, and the property information.

11. The analysis method according to claim 1, wherein
the second information obtained from the interior of the spring steel includes composition information on the spring steel.

12. The analysis method according to claim 1, wherein
the learning model learns a relationship between the material information, the material information including the first information obtained from the surface of the spring steel, the second information obtained from the interior of the spring steel, the mechanical properties of the spring steel, and the property information, the property information including the time-dependent properties of the spring steel.

13. A non-transitory computer-readable storage medium having a program stored thereon, the program causing a computer to:
provide first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and
obtain first property information on the first spring steel from the learning model.

14. An analysis device comprising:
a providing unit providing first material information obtained from a first spring steel of an analysis target to a learning model, the learning model having learned a relationship between material information and property information, the material information including first information obtained from a surface of spring steel and second information obtained from the interior of the spring steel, and the property information representing material properties of the spring steel, and
an acquisition unit obtaining first property information on the first spring steel from the learning model.

15. The analysis device according to claim 14, wherein
the second information obtained from the interior of the spring steel includes third information related to a periodic structure of the interior of the spring steel.

16. The analysis device according to claim 15, wherein
the third information related to a periodic structure is obtained from an XRD spectrum.

17. The analysis device according to claim 14, wherein
the second information obtained from the interior of the spring steel is at least one of dislocation density, solid-solution carbon content, retained γ, dislocation character, residual stress, and inclusion distribution.

18. The analysis device according to claim 14, wherein
the first information obtained from the surface of the spring steel includes observation information on the surface of the spring steel.

19. The analysis device according to claim 14, wherein
the first information obtained from the surface of the spring steel is at least one of crystal grain size, carbide morphology, and surface roughness.

20. The analysis device according to claim 14, wherein
the material properties of the spring steel are at least one of mechanical properties and time-dependent properties.

21. The analysis device according to claim 20, wherein
the mechanical properties include hardness, tensile properties, and toughness.

22. The analysis device according to claim 20, wherein
the time-dependent properties include strain and fatigue.

23. The analysis device according to claim 14, wherein
the learning model includes learning a relationship between the material information, the material information including the first information obtained from the surface of the spring steel, the second information obtained from the interior of the spring steel, the condition information at the time of manufacturing the spring steel, and the property information.

24. The analysis device according to claim 14, wherein
the information obtained from the interior of the spring steel includes composition information on the spring steel.

25. The analysis device according to claim 14, wherein
the learning model learns a relationship between the material information, the material information including the first information obtained from the surface of the spring steel, the second information obtained from the interior of the spring steel, the mechanical properties of the spring steel, and the property information, the property information including the time-dependent properties of the spring steel.
